(19) United States
(12) Patent Application Publication
(10) Pub. No.: US 2015/0216489 A1
(43) Pub. Date: Aug. 6, 2015

(54) EMERGENCY ACTIVATION OF A MEDICAL X-RAY IMAGING SYSTEM

(71) Applicant: KONINKLIJKE PHILIPS N.V., Eindhoven (NL)

(72) Inventor: Fransciscus Joannes Leonardus Everaerts, Weert (NL)

(21) Appl. No.: 14/430,319

(22) PCT Filed: Sep. 22, 2013
(86) PCT No.: PCT/IB2013/058735

§ 371 Date: Mar. 23, 2015

Related U.S. Application Data
(60) Provisional application No. 61/710,792, filed on Oct. 8, 2012.

(51) Int. Cl. A61B 6/12; A61B 19/00; A61B 17/00; A61B 6/00; A61B 6/10

(52) U.S. Cl. CPC A61B 6/12; A61B 6/54; A61B 6/107; A61B 6/461; A61B 17/00234; A61B 19/44; A61B 2017/00207; A61B 2017/00203; A61B 2017/00384

(57) ABSTRACT

The present invention relates to emergency activation of a medical X-ray imaging system. In order to provide a facilitated way of visualizing a lost item inside a patient, a medical X-ray imaging system (10) is provided that comprises an X-ray image acquisition device (12) with an X-ray source (14) and an X-ray detector (16), a control device (18), an emergency activation interface device (22), and a display device (24). The X-ray source is configured to generate X-ray radiation of various characteristics, and the X-ray detector is configured to detect the X-ray radiation after being at least partly attenuated by an object of interest. The control device is configured to control the generation of the X-ray radiation by the X-ray source, and to generate image data from the detected X-ray radiation. The display device is configured to display an image representing the detected X-ray radiation. Further, the emergency activation interface device is configured to start a generation of X-ray radiation for maximal visibility of the object of interest upon being brought into an emergency mode.

### FIELD OF THE INVENTION

The present invention relates to a medical X-ray imaging system, a method for viewing an object of interest in an emergency case, a computer program element, and a computer readable medium.

### BACKGROUND OF THE INVENTION

During interventions, such as minimal invasive interventions, X-ray imaging can be provided to monitor the current activity inside an object of interest, for example inside a patient. In certain applications, for example for restoring structural heart diseases, devices can be introduced inside the vascular system, for example stents, clips, umbrellas and other components that may be delivered via a guiding catheter or a guide wire. Upon introduction into the vascular system, it may happen that the device is released, on purpose or even without intention, and thus becomes loose. This may require immediate action to prevent the lost item from floating in the blood stream to an unwanted position with the possible result that a vessel becomes blocked (partly or completely). However, it has been shown that the surgeon is confronted with a situation, in which the operation of the X-ray imaging system becomes rather complex, in order to detect the lost device inside the vascular system. Of course, instead of the vascular system, also other organic systems, in particular tubular systems, may lead to similar situations.

### SUMMARY OF THE INVENTION

There may be a need to provide a facilitated way of visualizing a lost item inside an object such as a patient.

The object of the present invention is solved by the subject-matter of the independent claims, wherein further embodiments are incorporated in the dependent claims.

It should be noted that the following described aspects of the invention apply also for the medical X-ray imaging system, the method for viewing an object of interest in an emergency case, the computer program element, and the computer readable medium.

According to a first aspect of the present invention, a medical X-ray imaging system is provided, comprising an X-ray image acquisition device with an X-ray source and an X-ray detector. The system further comprises a control device, an emergency activation interface device, and a display device. The X-ray source is configured to generate X-ray radiation of various characteristics. The X-ray detector is configured to detect the X-ray radiation after being at least partly attenuated by an object of interest. The control device is configured to control the generation of the X-ray radiation by the X-ray source. The control device is also configured to generate image data from the detected X-ray radiation. The display device is configured to display an image representing the detected X-ray radiation. The emergency activation interface device is configured to start a generation of X-ray radiation for maximal visibility of the object of interest upon being brought into an emergency mode.

The X-ray source may be an X-ray tube, for example an X-ray tube with a rotating anode, or an X-ray tube with carbon nanotube (CNT) emitters. The X-ray detector may be a digital X-ray detector, also referred to as DXR, for direct or indirect X-ray radiation.

The emergency activation interface device may be provided as a crisis button or emergency press for X-ray, which is why it can also be referred to as emergency feature (emergency EPX, thereafter named: EPX). The EPX may be tuned to maximal visualization of the lost devices. It must be noted that dose is of less importance in an emergency case. The main focus lies on optimized visibility for an easy and fast retrieval of the lost device.

The device may have been lost during an interventional procedure, for example during a cardiovascular intervention, which has been interrupted for the activation of the X-ray radiation for maximal visibility. The device may also be an already implemented device, which, during an independently performed interventional procedure, becomes loose and starts floating around in the blood stream.

According to an exemplary embodiment, the X-ray radiation for maximal visibility relates to an emergency setting as a setting for maximal performance to retrieve a device inside the object. In the emergency mode, predetermined maximum dose values are overruled to allow the system's optimized viewing capacity irrespective of dose restrictions.

The dose values may relate to skin entry dose, overall dosage, and others. Of course, legally required dose restrictions, varying in different countries, may be maintained.

For example, in an emergency situation, the legally required maximal dose may be exceeded under certain fulfilled requirements. Often, a hospital physicist is involved. It may be needed to provide an “emergency” dose report where the system generates a report with additional parameters, like dose area product (DAP) and air kerma (referring to the initial kinetic energy of primary ionizing particles that are produced by an interaction of the incident uncharged radiation in a small volume of air when this is irradiated by an X-ray beam), and also arm angle, dose per arm position, radiation intensity per second, etc. are recorded and listed.

According to an exemplary embodiment, the X-ray radiation parameters for maximal visibility comprise at least one of the group of: i) setting for maximal X-ray dose, ii) maximal focal spot, and iii) predetermined degree of high dose in combination with adjusted minimal image processing.

For example, one way to maximal visibility is to provide maximum X-ray intensity. However, maximal visibility may be obtained by other means beyond maximum X-ray intensity. Examples are auto zoom (in order to retrieve small objects), correlation with patient body mass index (BMI), location in the body (skull, bone, versus soft tissue). Basically, all parameters that are tuned in an X-ray system can be addressed, for example in dependency of the predetermined settings of a certain interventional procedure. For example, for neuro surgery, different parameters can be identified for providing the respective maximal X-ray radiation detection, compared to cardiovascular interventions.

According to an exemplary embodiment, the generation of the X-ray radiation for maximal visibility depends on the device to be retrieved.

Since a modern X-ray system typically contains multiple screens, in one embodiment all of these screens can be applied to assist the physician. For example, on one screen the original image is displayed while on the second screen a zoomed image is displayed. Another option is to process the detector data such that per screen an image is displayed that is digitally enhanced. For example parameters like: contract, brightness and noise reduction can be altered in order to optimize the image to locate the device.

In another embodiment one screen displays the image that is optimized for locating the device. On this screen a pointer is overlaid. On the second screen an image that is optimized for tissue recognition is displayed with the same pointer. If so the case, the physician is assisted in locating the device while having guiding directions for a support device to remove the lost item.

In another embodiment both images are overlaid.

Before the intervention, the interventional type that will be used, or the different types or models that will be used, is/are entered into the system by an interface. When activating the emergency activation interface device, the interventional device type is considered. In case of different types, the activation of the emergency activation interface device may comprise the selection of one or more device types or categories.

According to an exemplary embodiment, the emergency activation interface device is provided as at least one of the group of: one or more separate button(s) or combination of buttons to be provided in the vicinity of an operation table, key or key combination on an interface for operating the X-ray imaging system, voice command to be recorded by a microphone device and detected by speech recognition provided by the control device, and gesture command to be recorded by a video camera device and detected by gesture recognition provided by the control device, and activation area on a touch sensitive display.

The emergency activation interface device can be brought into the emergency or activation mode, for example by touching or pressing one of the above. In the emergency or activation mode, the X-ray radiation for maximal visibility is triggered.

According to an exemplary embodiment, safety measurements are provided that are configured to detect between at least one predetermined registered state and an unregistered state. The safety measurements are further configured to restrict the start of the generation of X-ray radiation for maximal visibility to the detected registered state.

The safety measurements may be provided to prevent an unintentional activation of the X-ray radiation for maximal visibility. For example, the EPX can only be used during emergency situations, which may be supported by the safety measurements or safety measures.

According to an exemplary embodiment, the safety measurements comprise at least one of the group: password or code protection, key combination or key sequence required to be entered by two persons, pressing or activating of two or more buttons at the same time, presence of the clinical physicist of the hospital, which is detected by a staff monitoring device, and voice recognition of authorized persons.

A feedback device may be provided that provides a feedback signal to the user when the system is operating in the emergency mode. The feedback device may comprise at least one of the group of an audio feedback element, a visual feedback element, and a haptic feedback element.

The safety measurements may comprise a protocol device that generates a report when having operated in the emergency mode, wherein the report needs to be signed by the hospital physicist, for example.

According to a second aspect, a method for viewing an object of interest in an emergency case is provided, comprising the following steps:
a) bringing an emergency activation interface device into an emergency mode;
b) generating of X-ray radiation for maximal visibility of an object of interest;
c) detecting the X-ray radiation after being at least partly attenuated by the object of interest;
d) generating image data from the detected X-ray radiation; and
e) displaying an image representing the detected X-ray radiation.

According to an aspect of the present invention, a specially configured interface is provided such that, for example, the surgeon can simply activate the interface to start the respectively adapted image acquisition and image presentation for identifying the position/location of a lost device, for example, during an interventional procedure. Thus, by activating the interface, an image acquisition is started that is specifically configured for an optimal visibility of the device in order to provide device detection and retrieving of the device in a minimum amount of time, i.e. as fast as possible. Such emergency situation may occur in particular in relation with cardiac-related interventions. For example, for restoring structural and congenital cardiac defects, it is known to use minimal invasive techniques, in which devices are introduced, for example for closing openings, or for widening tubular structures. The devices may be self-expandable meshes, for example covered with a polymer material; another example are stents. In particular with self-expandable meshes, which are provided in place in the tubular system, such that blood cloth formation occurs inside the meshes, after several months it is expected that the implant is totally embedded and endotheliazed. Further, besides the introduction procedure of the device, the optimal positioning of the device is an important step, for which step imaging equipment is provided. However, tissue abnormalities that cannot be visualized, for example, may cause that, after the device is in place, the device may slip, reorient, and in some cases may start to float in the blood stream. A floating device could cause severe harm to the tissue and in worst case may block a vessel. Thus, an intermediate intervention or reaction is needed. But before the patient is rushed to an operation room for emergency surgery, the interventional cardiologist may try to retrieve the device. For example, a large sheet is introduced into the vascular system in combination with a biopsy catheter. After localization of the device, the device is pulled in the sheet and removed. In such an emergency situation where first the device inside the patient has to be found, which situation may be considered by a physician active in this field as “nightmare case”, the activation for maximal visualization of the lost device is started by the emergency button or emergency EPX. Therefore, the X-ray system operates under maximum performance in order to assist the physician for the emergency retrieval of the lost device. Although the activation is provided as simple as possible, safety measurements may be provided in order to prevent the EPX from being used during normal cases.

These and other aspects of the present invention will become apparent from and be elucidated with reference to the embodiments described hereinafter.

### BRIEF DESCRIPTION OF THE DRAWINGS

Exemplary embodiments of the invention will be described in the following with reference to the following drawings:

FIG. 1 schematically shows a medical X-ray imaging system;

FIG. 2 shows different embodiments of an emergency activation interface in FIGS. 2A, 2B, 2C, 2D, and 2E in respective schematic illustrations; and

FIG. 3 shows basic steps of a method for viewing an object of interest in an emergency case.

### DETAILED DESCRIPTION OF EMBODIMENTS

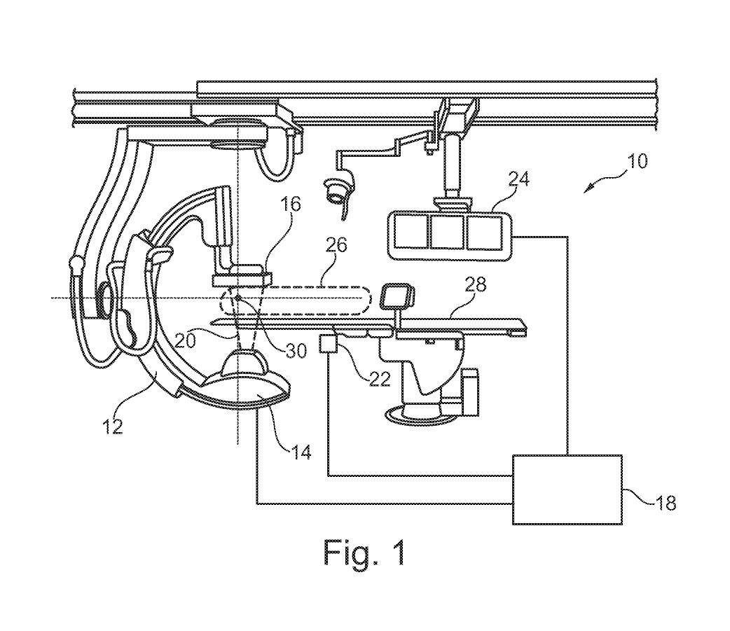

FIG. 1 shows a medical X-ray imaging system 10, comprising an X-ray image acquisition device 12 with an X-ray source 14 and an X-ray detector 16. Further, a control device 18 is provided, which is configured to control the generation of X-ray radiation 20 by the X-ray source 14. The control device 18 is further configured to generate image data from the detected X-ray radiation. Further, an emergency activation interface device 22 is provided, which will also be explained in more detail with reference to the following figures in different examples. Still further, a display device 24 is provided. The X-ray source 14 is configured to generate X-ray radiation of various characteristics, and the X-ray detector 16 is configured to detect the X-ray radiation after being at least partly attenuated by an object of interest, for example a patient 26 provided on a patient support 28, also referred to as patient table. The display device 24 is configured to display an image representing the detected X-ray radiation.

The X-ray imaging acquisition device 12 may be provided, as shown in FIG. 1, as a C-arm structure, where the X-ray source and the X-ray detector are mounted to opposing ends of a C-arm, which C-arm is movable mounted to a support (not further shown). However, according to a further example (not further shown), the medical X-ray imaging system 10 is provided as a fixed X-ray imaging system with fixedly mounted X-ray source and X-ray detector. In a still further example, the medical X-ray imaging system is provided as a CT system, for example with a rotating gantry (not further shown).

The emergency activation interface device 22 is configured to start a generation of X-ray radiation for maximal visibility of the object of interest upon being brought into an emergency mode.

The X-ray radiation for maximal visibility relates to an emergency setting as a setting for maximum performance to retrieve a device 30 inside the object, for example inside the patient 26. In the emergency mode, predetermined maximum dose values are overruled to allow the system to optimize the viewing capacity irrespective of dose restrictions. It must be noted that the X-ray radiation parameters for maximal visibility comprise the setting for maximum X-ray dose, but can also comprise, alternatively or in addition, parameters for maximum focal spot, and/or predetermined degree of high dose in combination with adjusted minimal image processing. Even one way to provide maximal visibility is the provision of maximum X-ray intensity, but the maximum visibility may also be obtained by other measures, such as auto zoom, or a correlation with the patient's characteristics, for example the BMI, or the location in the body. In particular, the generation of the X-ray radiation for maximal visibility may depend on the device that is to be retrieved. For example, before the intervention, an input is provided to inform the system about the interventional device or devices that is/are used during the intervention.

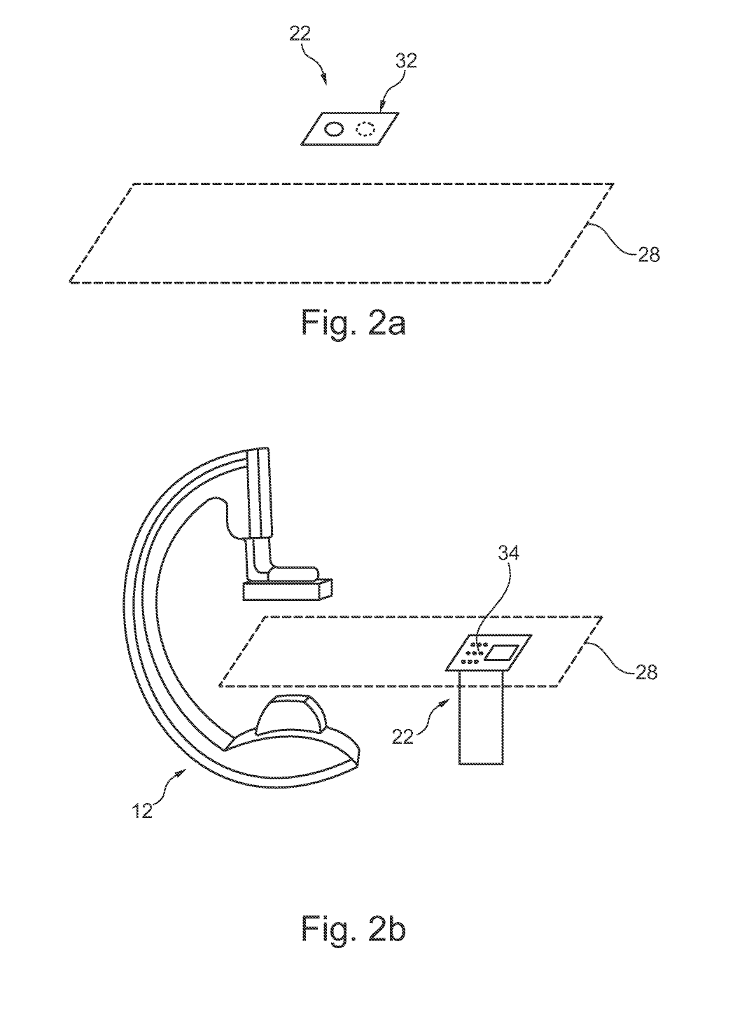

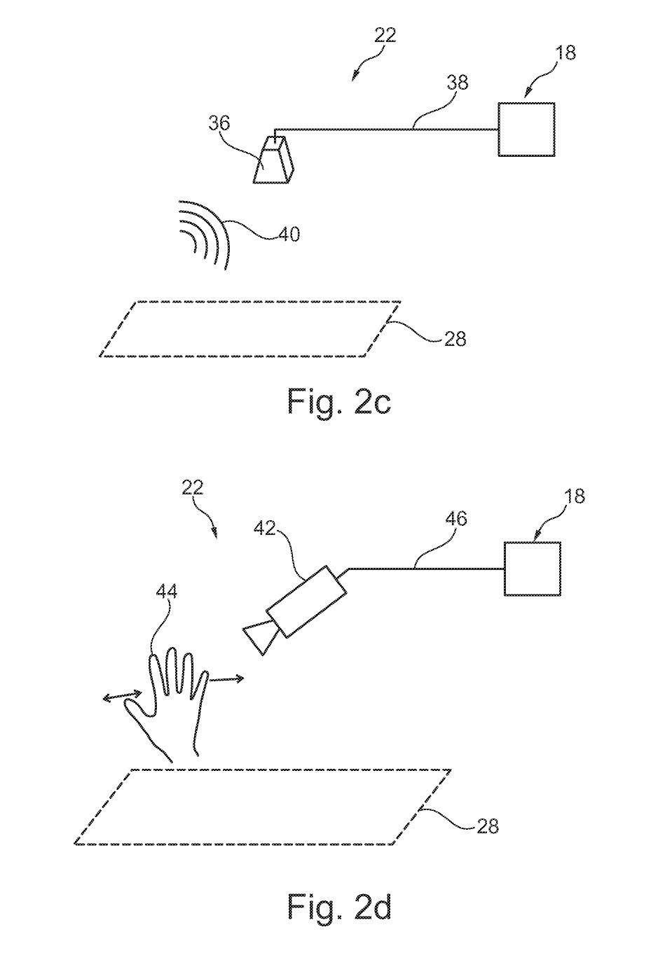

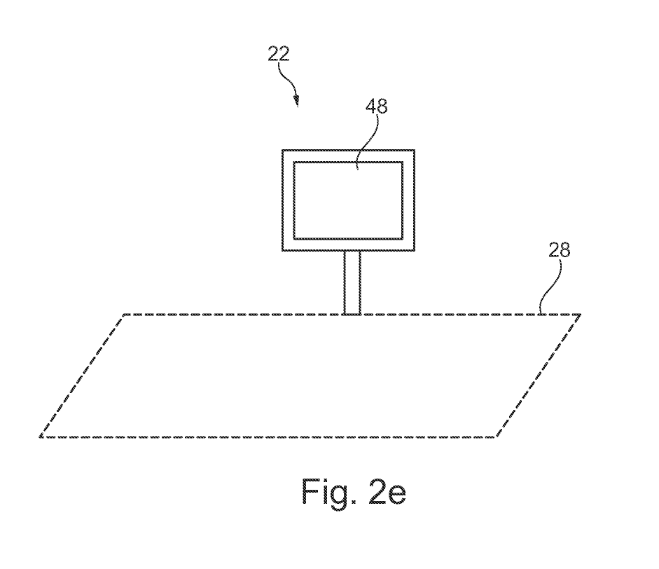

As shown in FIG. 2A, one or more separate button(s) or combination of buttons 32 may be provided in the vicinity of an operation table 28 as the emergency activation interface device 22.

As shown in FIG. 2B, a key or key combination on an interface 34 for operating the X-ray imaging system may be provided as the emergency activation interface device 22.

As shown in FIG. 2C, a microphone device 36 can be provided to record a voice command as the emergency activation interface device 22. The recorded voice command is detected, for example, by speech recognition by the control device 18, which is indicated by a connecting line 38. The voice command is indicated with sound wave symbol 40.

As shown in FIG. 2D, a video camera device 42 may be provided to record a gesture command, indicated with a waving hand symbol 44, which is detected by gesture recognition provided by the control device 18, which is indicated by connecting line 46.

As shown in FIG. 2E, an activation area on a touch sensitive display 48 may be provided as the emergency activation interface device 22.

According to a further example (not further shown), safety measurements are provided that are configured to detect between at least one predetermined registered state and an unregistered state. The safety measurements are further configured to restrict the start of the generation of X-ray radiation of X-ray radiation for maximal visibility to the detected registered state. In other words, the safety measurements are provided to prevent an unintentional activation of the X-ray radiation for maximal visibility. The EPX can thus only be used during an emergency situation, which may be provided by safety measurements comprising at least one of the group of: password or code protection, key combination or key sequence required to be entered by two persons, pressing or activating of two or more buttons at the same time, presence of the clinical physicist of the hospital, which is detected by a staff monitoring device, and voice recognition of authorized persons.

According to a further example (not shown), a feedback device is provided for giving a feedback signal to the user when the system is operating in the emergency mode. For example, an audio feedback element, a visual feedback element, and/or a haptic feedback element is provided to indicate that the system is operating for maximal visibility of a lost device.

Further, a protocol device that generates a report when having operated in the emergency mode can be part of the safety measurements.

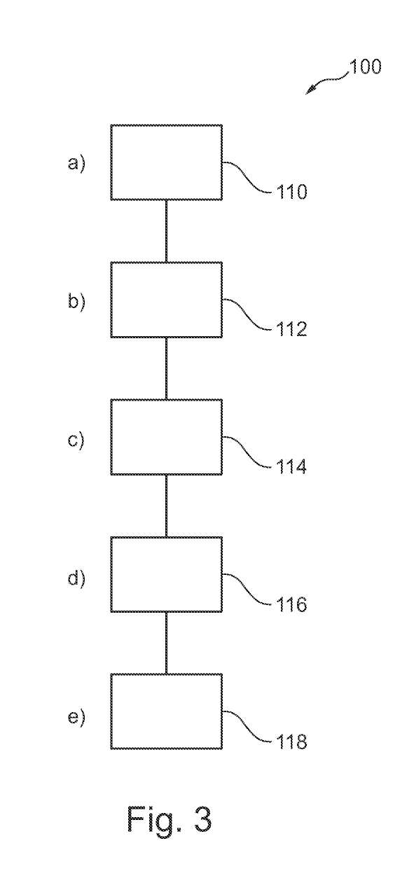

FIG. 3 shows a method 100 for viewing an object of interest in an emergency case, which method comprises the following steps: In a first step 110, an emergency activation interface device is brought into an emergency mode. In a second step 112, X-ray radiation for maximal visibility of an object of interest is generated. In a third step 114, the X-ray radiation is detected after being at least partly attenuated by the object of interest. In a fourth step 116, image data from the detected X-ray radiation is generated. In a fifth step 118, an image representing the detected X-ray radiation is displayed. The first step 110 is also referred to as step a), the second step 112 as step b), the third step 14 as step c), the fourth step 116 as step d), and the fifth step 118 as step e).

According to a further example, the emergency activation interface device is brought into an emergency mode by activating one of the above described examples of the emergency activation interface device, for example as shown in FIGS. 2A to 2E.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A medical X-ray imaging system, comprising:
an X-ray image acquisition device with an X-ray source and an X-ray detector,
a control device;
an emergency activation interface device; and
a display device;
wherein the X-ray source is configured to generate X-ray radiation of various characteristics; and wherein the X-ray detector is configured to detect the X-ray radiation after being at least partly attenuated by an object of interest;
wherein the control device is configured to control the generation of the X-ray radiation by the X-ray source; and to generate image data from the detected X-ray radiation;
wherein the display device is configured to display an image representing the detected X-ray radiation; and
wherein the emergency activation interface device is configured to be brought into an emergency mode wherein predetermined maximum dose values are overruled to allow an optimized viewing capacity for retrieving a device lost inside the object of interest, irrespective of dose restrictions.

2. (canceled)

3. Imaging system according to claim 1, wherein the X-ray radiation parameters for maximal visibility comprise at least one of the group of:
i) setting for maximal X-ray dose;
ii) maximal focal spot; and
iii) predetermined degree of high dose in combination with adjusted minimal image processing.

4. Imaging system according to claim 1, further comprising an interface for entering a type or model of an interventional device that will be used, wherein the interventional device type or model is considered when activating the emergency mode.

5. Imaging system according to claim 1, wherein the emergency activation interface device is provided as at least one of the group of:
one or more separate button(s) or combination of buttons to be provided in the vicinity of an operation table;
key or key combination on an interface for operating the X-ray imaging system;
voice command to be recorded by a microphone device and detected by speech recognition provided by the control device;
gesture command to be recorded by a video camera device and detected by gesture recognition provided by the control device; and
activation area on a touch sensitive display.

6. Imaging system according to claim 1, wherein safety measurements are provided that are configured to detect between at least one predetermined registered state and an unregistered state; and to restrict the start of the generation of X-ray radiation for maximal visibility to the detected registered state.

7. Imaging system according to claim 6, wherein the safety measurements comprise at least one of the group of the following:
password or code protection;
key combination or key sequence required to be entered by two persons;
pressing or activating of two or more buttons at the same time;
presence of the clinical physicist of the hospital, which is detected by a staff monitoring device; and
voice recognition of authorized persons.

8. A method for viewing an object of interest in an emergency case, comprising the following steps:
a) bringing an emergency activation interface device into an emergency mode wherein predetermined maximum dose values are overruled to allow an optimized viewing capacity for retrieving a device lost inside the object of interest, irrespective of dose restrictions;
b) generating of X-ray radiation;
c) detecting the X-ray radiation after being at least partly attenuated by the object of interest;
d) generating image data from the detected X-ray radiation; and
e) displaying an image representing the detected X-ray radiation.

9. A computer program element for controlling an apparatus according to claim 1, which, when being executed by a processing unit, is adapted to perform the method step of claim 8.

10. A computer readable medium having stored the program element of claim 9.
